(19) United States
(12) Patent Application Publication
(10) Pub. No.: US 2003/0027805 A1
(43) Pub. Date: Feb. 6, 2003

(54) Use of 1,4-androstadienedione as a method of increasing levels of the anabolic/androgenic steroid boldenone in humans

(76) Inventor: William Charles Llewellyn, Sound Beach, NY (US)

Correspondence Address: William Llewellyn, PO Box 1162, Sound Beach, NY, 11789, US

(21) Appl. No.: 10/202,666

(22) Filed: Jul. 25, 2002

Related U.S. Application Data
(63) Continuation-in-part of application No. 09/641,526, filed on Aug. 21, 2000.

(51) Int. Cl. A61K 31/57

(52) U.S. Cl. 514/177

(57) ABSTRACT

This invention discloses a method of administering 1,4-androstadienedione as a means of increasing boldenone levels in humans. As men age, a decline in androgenic hormone levels is typically noted, possibly resulting in muscle mass, bone density and energy loss. Various methods have therefore been developed to supplement androgens for men with declining levels. This invention is an improvement over the previously suggested steroid precursors, in that androstadienedione displays superior oral activity. This may be a very advantageous trait for men seeking the an orally active form of androgen replacement.

### CROSS-REFERENCE TO RELATED APPLICATIONS

Continuation in-part of application Ser. No. 09/641,526, now abandoned.

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

Androgens are responsible for the development and maintenance of male sexual characteristics, including external virilization, sexual maturity at puberty, spermatogenesis, sexual behavior/libido and erectile functioning. They also supports bone and muscle tissue growth, and remains vital to ones health and well being throughout life. After physical maturity, men often notice a slow decline in the level of androgens produced by the body. Dubbed andropause, subnormal androgen levels can lead to a decline in muscle mass, libido, sexual functioning and overall sense of well being later in life. In many instances this indicates a need for some form of androgen replacement.

This invention relates a method of administering 1,4-androstadiene-3,17-dione, as a means of obtaining an anabolically or androgenically active serum level of boldenone in humans. Boldenone is an anabolic/androgenic steroid structurally similar to the primary male androgen testosterone. Although similar in structure however, its action in the human body is measurably different from that of testosterone. The primary difference stems from the fact that boldenone is much more slowly aromatized to estradiol, and as a result this hormone displays less estrogenic activity than testosterone. Boldenone is also less active at promoting androgenic activity in target tissues where the 5-alpha reductase enzyme normally converts testosterone to its more potent form of dihydrotestosterone, as it is highly resistant to interaction with this enzyme. Being an active anabolic/androgenic steroid however, the activity of boldenone is still capable of inducing the expected benefits of androgen replacement on mood, sexual function, bone and muscle, as all such effects are mediated by the cellular androgen receptor.

A number of methods have been developed to restore androgen concentration in humans with declining levels. Several injectable esterified testosterone preparations have been fashioned that allow a slow release of hormone into the blood stream over the course of several days to weeks for example, however all provide inconsistent dosing as there is great variance in hormone release from the site of injection, such that a short supraphysiological rush may eventually be followed by days of subnormal hormone concentrations. The buildup of estrogens due to the natural process of aromatization may exaggerate the side effects to such medication as well, particularly at times when testosterone levels are abnormally high, as supraphysiological levels of estrogens in the male body have been linked to gynecomastia (female breast tissue development), water retention and edema, and increased fat deposition. More basically, recent studies have made clear that both androgens and estrogens play synergistic roles in the promotion of benign prostatic hypertrophy (BPH). This suggests that a readily aromatizable androgen such as testosterone may be less than ideal for use in older men at risk for such disease.

Also a number of synthetic oral androgen derivatives have been developed including methyltestosterone, fluoxymesterone and stanozolol. All such compounds are alkylated at the 17^{th} carbon position (alpha orientation), an alteration that inhibits reduction of the steroid to inactive 17-ketosteroid form. While this greatly improves oral bioavailability of the compound, this alteration has also been shown to place stress on the liver, in some instances resulting in organ damage. Although the use of a c-17 alpha alkylated oral androgen may prove much more comfortable for the patient in terms of dosing and control over blood hormone level compared to an injectable preparation, the possible risk of developing complications with liver functions may make them much less useful for androgen replacement compared to injectable preparations, particularly for extended periods of therapy.

In searching for a less toxic, more reliable oral alternative for androgen replacement the use of androgen precursor hormones have been suggested. U.S. Pat. No. 5,578,588 to Mattern et al. relates a method of using a precursor hormone, namely androstenedione, as a means of increasing testosterone levels. U.S. Pat. No. 5,880,117 to Patrick Arnold. relates a method of using the precursor hormone 4-androstenediol as a means of increasing testosterone levels in humans. U.S. Pat. No. 6,391,868 to Arnold similarly relates a method of using 5-alpha-androst-1-en-3-one for increasing levels of the anabolic/androgenic steroid 17-beta-hydroxy-5-alpha-androst-1-en-3-one in humans. U.S. Pat. No. 6,262,436 to Llewellyn discloses the method of using 5-alpha-androstanedione or 5-alpha-androstanediol to increase levels of dihydrotestosterone in humans. The pharmacokinetics of administering such precursors is such that hormone concentrations of active hormone (testosterone) peak within 90 minutes, and subsequently decline over a period of three to four hours. This more closely resembles the natural pulsating pattern in which the body releases testosterone, and avoids the prolonged peaks and troughs noted with use of esterified injectable hormone preparations.

### BRIEF SUMMARY OF THE INVENTION

Prior art relates novel methods of using several different precursor hormones to active anabolic/androgenic steroids as a means of increasing androgen levels in humans. Although the suggested practice of using a precursor to an active hormone seems quite sound, the target hormone of replacement may be less than ideal in certain cases, particularly in those where a hormone with low but measurable estrogen conversion is desirable or a more orally precursor therapy is needed. The problem of the present invention is therefore to provide another naturally occurring androgenic hormone precursor that can be used to replace androgen action in humans but displays a low level of undesirable estrogenic activity and high oral potency. According to the invention this problem is solved by the use of 1,4-androstadienedione, a direct precursor to the anabolic/androgenic steroid boldenone. This precursor of boldenone is ideal because it is natural, non-toxic, quickly metabolized to active form after oral administration, less actively metabolized to estradiol in the human body than testosterone, and displays a high tendency to convert to active form.

### BRIEF DESCRIPTION OF THE SEVERAL VIEWS OF THE DRAWING

Not Applicable

### DETAILED DESCRIPTION OF THE INVENTION

The term androstadienedione refers to the chemical 1,4-androstadiene-3,17-dione. Androstadienedione has been identified as a direct metabolite of boldenone in human placental, uterine, testicular, adrenal and nervous system tissues. The compound androstadienedione has additionally been shown to occur in nature, as was demonstrated in a study by Miller et al. (J. Biol. Chem 220:221-5 1956) which recovered this hormone in the feces of a pregnant dairy cow. Androstadienedione acts as an efficient precursor to boldenone in humans, converting to active form via the 17beta-hydroxysteroid-dehydrogenase enzyme.

Human tests carried out by Ferdinando Galleti and Rinaldo Gardi (Steroids: July 1971; 39-50) have fundamentally proven the in-vivo transformation of androstadienedione to boldenone. In this study it was demonstrated that within 24 hours after oral administration of 100 mg androstadienedione, urinary recovery of boldenone conjugates in the two subjects of this investigation was measured to be 7.1% and 11.1% of the initial dosage. The additional isolation of other 17-beta hydroxysteroids increases the total recovery of 17-OH steroids to 18.4% and 21.2% of the given dose in the respective subjects. The authors made note that the urinary recovery of 17-hydroxysteroids was unusually high compared to what is reported after administration of testosterone or androstenedione, which are excreted in 17-hydroxy form only in very small amounts after oral dosing.

In a later but similar study with 1-androstenedione (J Steroid Biochem (3) 933), the precursor hormone disclosed in U.S. Pat. No. 6,391,868 to Arnold, these same authors commented that 1,4-androstadienedione excreted far more 17beta-hydroxysteoid metabolites than this hormone in comparison. Based on the belief that the high urinary recovery of hydroxysteroids would relate positively to the oral bioavailability of this compound as a prohormone to boldenone, this suggested to this inventor that androstadienedione would be superior to other steroidal precursors hormones, in that it should display a much higher level of oral potency than those disclosed in the prior art.

After learning of the high conversion of androstadienedione to boldenone in vivo, plus the suitability of boldenone as a target for androgen replacement, it became the focus of this invention that the advantages of androgen replacement can be achieved with 1,4-androstadienedione. In an effort to prove this theory a clinical study was therefore undertaken by the inventor. Specifically, it was the intention of the inventor to prove that 1,4-androstadienedione would act as an effective in-vivo peroral boldenone precursor in man.

The effective oral daily dosage of androstadienedione should be 25 mg to 500 mg. Due to the rapidity in which the discussed compound is metabolized in the body the total daily dosage can be further subdivided for a more sustained blood hormone concentration, with 3-5 applications per day being most preferred. In addition to peroral use, androstadienedione can be effectively administered by several other routes including transdermal, sublingual or intranasal. In order to increase bioavailability during sublingual or intranasal administration the target hormone can additionally be complexed with a cyclodextrin such as hydroxypropyl-beta-cyclodextrin.

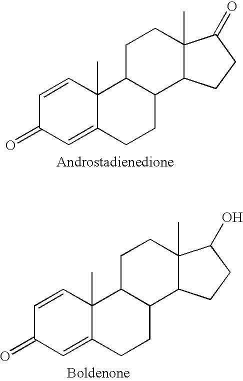

I claim:

1. A method for increasing boldenone levels in humans by administering an effective amount of 1,4-androstadiene-3,17-dione.

2. A method according to claim 1, wherein the method of administration is peroral.

3. The method of claim 1 wherein said administration is selected from the group consisting of transdermal, intranasal, and sublingual.

4. A method according to claim 1 wherein a daily dosage of 25 mg to 1000 mg is used.
